(19) United States
(12) Patent Application Publication
(10) Pub. No.: US 2026/0069187 A1
(43) Pub. Date: Mar. 12, 2026

(54) CAPACITOR SIZE REDUCTION FOR HIGH-PASS FREQUENCY IN ANALOG FRONT-END

(71) Applicant: STMicroelectronics International N.V., Geneva (CH)

(72) Inventors: Pasquale Biancolillo, Pedara (IT); Antonio Cicero, Palermo (IT); Angelo Recchia, Fasano (IT)

(21) Appl. No.: 18/828,522

(22) Filed: Sep. 9, 2024

(51) Int. Cl. A61B 5/308; A61B 5/053; A61B 5/304; H03F 3/45

(52) U.S. Cl. CPC A61B 5/308; A61B 5/053; A61B 5/304; H03F 3/45475; H03F 2200/129; H03F 2200/261

(57) ABSTRACT

According to an embodiment, a biopotential measurement system includes an analog front-end and digital circuits. The analog front-end circuit includes a sensing electrode, a forward amplifier, and a feedback amplifier with an integration capacitor. A feature is the attenuation circuit between the forward and feedback amplifiers, which provides an attenuation factor determining the integration capacitor's value for achieving the desired high-pass corner frequency. With configurable attenuation factors, the system can process different biopotential signals. Multiple analog front-end circuits can be used to process different signals simultaneously. The digital circuit includes a processor for signal processing, dynamic adjustment of attenuation factors, and anomaly detection. Additional components like switched capacitors, pseudo-resistors, and multiplexers can enhance the system's functionality. The design allows flexible, multi-parameter physiological monitoring with adjustable frequency responses and gain settings.

### TECHNICAL FIELD

The present disclosure generally relates to electronic devices and, in particular embodiments, to reducing capacitor size for high-pass frequency in an analog front-end.

### BACKGROUND

Various devices can measure biosignals and physiological signals in medical applications, providing data for healthcare professionals and patients. These devices range from sophisticated medical equipment in hospitals to portable and wearable devices that allow continuous everyday monitoring.

Medical facilities can utilize specialized equipment to capture detailed biosignals such as electrocardiograms (ECG), photoplethysmograms (PPG), bioelectrical impedance analysis (BIA), and galvanic skin response/electrodermal activity (GSR/EDA). These devices often feature integrated circuits combining analog and digital signal processing components.

Further, portable and wearable devices, including smartwatches and smartphones, are increasingly used to monitor various health parameters outside clinical settings. These consumer-grade devices often incorporate sensors for heart rate, activity levels, and even basic ECG measurements.

Moreover, remote monitoring devices enable healthcare providers to track patient's vital signs from a distance, improving care for those with chronic conditions. Environmental devices measure factors impacting health, such as air quality or UV exposure.

Across these applications, the trend has been towards more integrated, multi-functional systems that can simultaneously process and analyze multiple biosignals, offering a comprehensive view of an individual's health status.

### SUMMARY

Technical advantages are generally achieved by embodiments of this disclosure, which describe the reduction of capacitor size for high-pass frequency in an analog front-end.

A first aspect relates to a biopotential measurement system, comprising a sensing electrode configured to detect a biopotential signal; an analog front-end circuit coupled to the sensing electrode, the analog front-end circuit comprising a forward amplifier configured to amplify the biopotential signal; a feedback amplifier configured to integrate an output of the forward amplifier and set a high-pass corner frequency of the analog front-end circuit, the feedback amplifier comprising an integration capacitor and an amplifier; an attenuation circuit coupled between an output of the forward amplifier and an input of the feedback amplifier, wherein the attenuation circuit provides an attenuation factor that determines a capacitance value of the integration capacitor for achieving the high-pass corner frequency; and a digital circuit coupled to the analog front-end circuit, the digital circuit comprising a processor configured to process the amplified biopotential signal.

A second aspect relates to an analog front-end circuit for biopotential measurement, the analog front-end circuit comprising a forward amplifier configured to amplify a biopotential signal; a feedback amplifier configured to integrate an output of the forward amplifier and set a high-pass corner frequency of the analog front-end circuit, the feedback amplifier comprising an integration capacitor and an amplifier; an attenuation circuit coupled between an output of the forward amplifier and an input of the feedback amplifier, wherein the attenuation circuit provides an attenuation factor that determines a capacitance value of the integration capacitor for achieving the high-pass corner frequency; and a circuit arranged between the attenuation circuit and a common node between an inverting input of the amplifier of the feedback amplifier and the integration capacitor, the circuit comprising switched capacitors or a pseudo-resistor.

A third aspect relates to a biopotential measurement system, comprising a first analog front-end circuit configured to process a first biopotential signal, the first analog front-end circuit comprising a first forward amplifier, a first feedback amplifier comprising a first integration capacitor and a first amplifier, and a first attenuation circuit coupled between an output of the first forward amplifier and an input of the first feedback amplifier, wherein the first attenuation circuit provides a first attenuation factor, a second analog front-end circuit configured to process a second biopotential signal, the second analog front-end circuit comprising a second forward amplifier, a second feedback amplifier comprising a second integration capacitor and a second amplifier, and a second attenuation circuit coupled between an output of the second forward amplifier and an input of the second feedback amplifier, wherein the second attenuation circuit provides a second attenuation factor; and a digital circuit comprising a processor coupled to outputs of the first and second analog front-end circuits, the processor configured to dynamically adjust the first and second attenuation factors based on characteristics of the first and second biopotential signals.

Embodiments can be implemented in hardware, software, or any combination thereof.

### BRIEF DESCRIPTION OF THE DRAWINGS

For a more complete understanding of the present disclosure and the advantages thereof, reference is now made to the following descriptions taken in conjunction with the accompanying drawings, in which:

FIG. 1 is a simplified block diagram of an embodiment medical sensor;

FIG. 2 is a schematic of an embodiment analog front-end (AFE) system;

FIG. 3 is a schematic of an embodiment feedback integrator circuit;

FIG. 4 is a simplified schematic of an embodiment circuit;

FIG. 5 is a simplified schematic of an embodiment circuit; and

FIG. 6 is a block diagram of an embodiment medical device for biosignal measurements.

### DETAILED DESCRIPTION OF ILLUSTRATIVE EMBODIMENTS

This disclosure provides many applicable inventive concepts that can be embodied in a wide variety of specific contexts. The particular embodiments are merely illustrative of specific configurations and do not limit the scope of the claimed embodiments. Features from different embodiments may be combined to form further embodiments unless noted otherwise. Various embodiments are illustrated in the accompanying drawing figures, where identical components and elements are identified by the same reference number, and repetitive descriptions are omitted for brevity.

Variations or modifications described in one of the embodiments may also apply to others. Further, various changes, substitutions, and alterations can be made herein without departing from the spirit and scope of this disclosure as defined by the appended claims.

While the inventive aspects are described primarily in the context of medical applications and medical equipment, it should also be appreciated that the inventive aspects may also apply to other applications and industries. The principles and techniques described herein for reducing integration capacitance and improving analog front-end performance can be beneficial in various fields requiring precise, low-noise signal processing. For example, the concepts could be applied in fields requiring precise, low-noise signal processing, such as environmental sensing, industrial automation, geophysical instrumentation, and portable consumer electronics.

The disclosure presents an approach to addressing the challenge of large integration capacitance in analog front-end circuits for biosignal measurements, particularly in electrocardiogram (ECG) and bioelectrical impedance analysis (BIA) applications. In embodiments, an attenuation circuit is introduced in the feedback path of the analog front-end, which allows for a significant reduction in the size of the integration capacitor. The modification enables the achievement of very low high-pass frequencies, advantageous for accurate biosignal measurements, while maintaining a fully integrated solution.

Advantageously, the proposed approach allows for a significant reduction in the required capacitance, potentially decreasing it from the nanofarads range to approximately 10 picofarads. The reduction translates directly to significant chip area savings and potential improvements in circuit performance. Further, the design introduces flexibility, allowing the same circuit structure for ECG and BIA applications by adjusting the attenuation ratio. The attenuation factor can be dynamically adjusted during different operational phases, optimizing performance for specific applications or operating conditions. The proposed approach addresses critical challenges in integrated circuit design for medical devices, offering a solution that is more area-efficient, cost-effective, and versatile than conventional approaches. These and additional details are further detailed below.

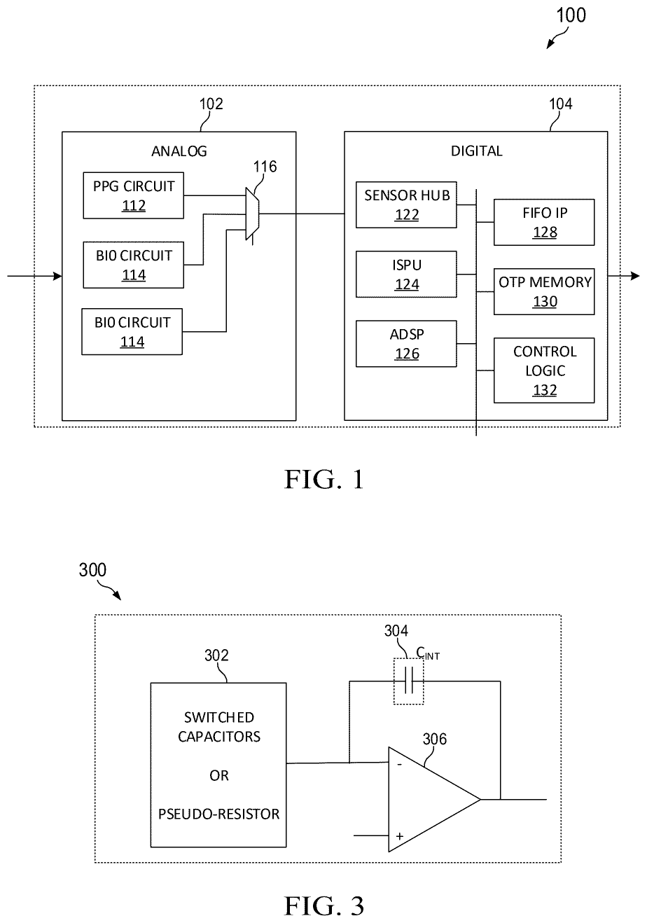

FIG. 1 illustrates a simplified block diagram of an embodiment medical sensor 100. Medical sensor 100 includes two main functional circuits: an analog circuit 102 and a digital circuit 104, which may (or may not) be arranged as shown. It should be appreciated that medical sensor 100 may include further analog and digital circuits.

The analog front-end can be optimized for low-noise performance and power efficiency in dealing with small amplitude signals. At the same time, the digital backend can provide the flexibility and computational power needed for advanced signal analysis and interpretation.

Analog circuit 102 receives the input signal, which in the context of a medical sensor may be a biosignal such as an electrocardiogram (ECG), photoplethysmogram (PPG), bioelectrical impedance analysis (BIA) signal, or galvanic skin response/electrodermal activity (GSR/EDA) signal. In embodiments, analog circuit 102 conditions and amplifies the typically weak biosignals before they are digitally processed.

As shown, analog circuit 102 includes a photoplethysmogram (PPG) circuit 112 and a pair of biopotential (BIO) circuits 114, which may (or may not) be arranged as shown. Analog circuit 102 may include additional components that are not shown, such as additional PPG or BIO circuits. It should be appreciated that analog circuit 102 may include a single biopotential circuit.

In embodiments, the photoplethysmogram (PPG) circuit 112 is configured to drive the PPG sensing components (e.g., emitters), collect the data measured by the PPG sensing components (e.g., detectors), and convert the PPG signals from PPG sensors to a digital signal. The photoplethysmogram (PPG) circuit 112 can include one or more multiplexers to select between photodiode inputs, transimpedance amplifiers (TIAs) to convert the photodiode current to a voltage, and low-noise amplifier (LNA) buffers to further amplify and condition the PPG signals.

Further, biopotential (BIO) circuit 114 is configured to process, for example, bioelectrical impedance analysis (BIA) signals, electrocardiogram (ECG) signals, galvanic skin response/electrodermal activity (GSR/EDA) signals from various sensors of the medical sensor 100. Biopotential (BIO) circuit 114 can include one or more multiplexers to select between the different input types, chopper-stabilized implementation amplifiers to provide low-noise, high-gain amplification of the typically weak biosignals, high-pass and low-pass filters to remove DC offsets and high-frequency noise.

Analog circuit 102 may include a multiplexer 116 that allows a selection between the PPG signal path (i.e., the output of the photoplethysmogram (PPG) circuit 112) and the BIO signal paths (i.e., the output of the biopotential (BIO) circuit 114), enabling, for example, time-division multiplexing of the different biosignals.

The analog circuit 102 may include additional components, such as analog-to-digital converters (ADCs) (e.g., high-resolution capacitive successive approximation register (SAR) ADCs) to convert analog signals to digital signals, voltage references and regulators for stability and accurate operation of the analog components, and oscillators to provide clock signals for the chopper amplifiers and other time-dependent circuits.

In embodiments, the output of the analog circuit 102, through the multiplexer 116, feeds into the digital circuit 104. In embodiments, digital circuit 104 is configured to process the digitized signal from analog circuit 102. In embodiments, the digital signal processing can include various digital signal processing techniques, data analysis, and algorithm implementation to extract relevant health information from biosignals.

Digital circuit 104 can include a sensor hub IP core circuit 122, an advanced intelligent signal processing unit (ISPU) 124, an advanced digital signal processor (ADSP) 126, a first-in-first-out (FIFO) IP core circuit 128, a one-time programmable (OTP) memory 130, and a control logic cells 132, which may (or may not) be arranged as shown. Digital circuit 104 may include additional components not shown, such as microprocessors, digital signal processors (DSPs), memory, and communication interfaces.

By integrating these specialized IP cores, digital circuit 104 can perform significant signal processing and analysis on-chip. The distributed processing approach reduces the computational load on any external host processor, minimizes power consumption, and enables faster response times. It also allows for on-sensor analytics, enabling real-time health monitoring and alerts without constant communication with an external system.

Sensor hub IP 122 can read external sensors using, for example, an I2C master protocol, enabling data collection in FIFO IP core circuit 128 and embedded elaboration also on the external data domain. It can also manage data collection and store it, for example, in the FIFO IP core circuit 128, and perform initial data formatting or simple preprocessing tasks.

In embodiments, the advanced ISPU 124 executes complex signal processing algorithms and directly runs machine learning or artificial intelligence models on the sensor. The “edge computing” capability can significantly reduce the amount of data that needs to be sent to an external processor or cloud service. In embodiments, the advanced ISPU 124 is a 32-bit core optimized for ultra-low-power operation while maintaining high performance.

In embodiments, the ADSP 126 is optimized to perform specific digital signal processing (DSP) functions. It can handle tasks like filtering, frequency analysis, or feature extraction from biosignals.

In embodiments, the FIFO IP core circuit 128 oversees data collection from multiple sources. The data can include data from the internal analog-to-digital converters, external sensors read by the sensor hub IP core circuit 122, and metadata or processed results generated by the ADSP 126 or the ISPU 124. The FIFO management ensures smooth data flow and helps prevent data loss during peak processing periods. In embodiments, data collected in the FIFO IP core circuit 128 is externally accessible. In embodiments, to optimize data collection and connection with an external memory or device, FIFO IP core circuit 128 allows the reading of the contents stored within when the FIFO is full.

In embodiments, OTP memory 130 stores critical calibration data (e.g., trimming data), security keys, or firmware that needs to be retained even when power is removed from the medical sensor 100.

In embodiments, the control logic cells 132 manage the various operation phases for the analog circuit 102. For example, the control logic cells 132 can control the timing of the chopper-stabilized amplifier, manage gain settings, or coordinate the operation of various filters in the analog front-end.

In embodiments, the output from digital circuit 104 represents the final processed signal or derived health information, which may be used for display, storage, or transmission to other devices or systems for further analysis or monitoring.

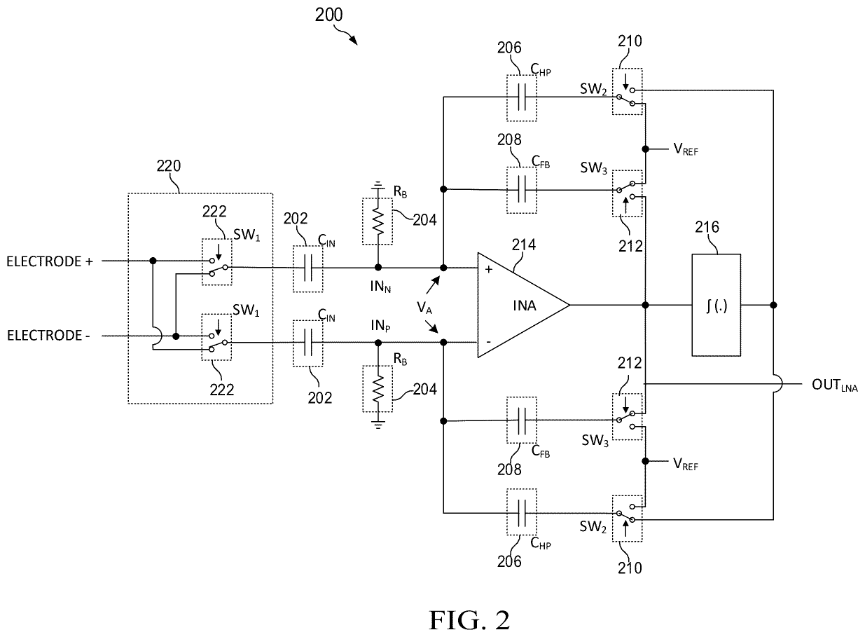

FIG. 2 illustrates a schematic of an embodiment analog front-end (AFE) system 200. AFE system 200 is configured to amplify and condition weak biosignals, such as those from ECG and BIA measurements. In embodiments, AFE system 200 employs a chopper-stabilized architecture to minimize low-frequency noise and offset, which can be advantageous for accurately detecting small physiological signals. Capacitive feedback and chopper stabilization enable improved noise performance and offset rejection, which is advantageous for detecting the microvolt-level signals typical in biosensing applications.

AFE system 200 includes an input chopper circuit 220, input capacitors (C_{IN}) 202, bias resistors (R_{B}) 204, high-pass capacitors (C_{HP}) 206, feedback capacitors (C_{FB}) 208, second switches (SW₂) 210, third switches (SW₃) 212, an instrumentation amplifier 214, and a feedback integrator circuit 216, which may (or may not) be arranged as shown. AFE system 200 may include additional components that are not shown, such as a dedicated controller and memory.

Input chopper circuit 220 includes first switches (SW₁) 222, which implement the chopping function at the input of the AFE system 200. In embodiments, the related de-chopper circuit is implemented internally to the instrumentation amplifier 214; however, it should be appreciated that the related de-chopper circuit may be implemented externally to the instrumentation amplifier 214 in embodiments.

The input chopper circuit 220 is configured to periodically reverse the electrode inputs (electrode + and electrode −) at the inverting and non-inverting inputs of the instrumentation amplifier 214. In embodiments, the switching mechanism is controlled through a controller internal to the AFE system 200 or a processor external to the AFE system 200.

The input stage of AFE system 200 includes input capacitors (C_{IN}) 202 that couple the electrode signals to the circuit while blocking DC components. In embodiments, input capacitors (C_{IN}) 202 are sized to provide a high input impedance at frequencies of interest, preventing the loading of the electrode-tissue interface. In embodiments, the summing node (VA) of the instrumentation amplifier 214 receives the differential signal input scaled by the input capacitors (C_{IN}) 202, balanced by two single-ended feedback networks.

Bias resistors (R_{B}) 204 provide a DC bias path for the input of the instrumentation amplifier 214. The bias resistors (R_{B}) 204 ensure minimal loading on the input signal while maintaining a stable DC operating point for the inputs of the instrumentation amplifier 214. Bias resistors (R_{B}) 204 can be implemented using, for example, standard high-value resistors, pseudo-resistors (i.e., metal-oxide-semiconductor (MOS) transistors in subthreshold), or other specialized structures.

The instrumentation amplifier 214 provides high-gain, low-noise amplification of the differential input signal. The instrumentation amplifier 214 modulates the input signal to a higher frequency, amplifies it, and then demodulates it back to the baseband, reducing 1/f noise (i.e., flicker or pink noise) and DC offset.

In embodiments, instrumentation amplifier 214 includes an amplifier stage, an output chopper for demodulation, and a low-pass filter to remove high-frequency components. The result is an amplifier with low offset voltage and drift, low-frequency noise performance, and a high common-mode rejection ratio.

In embodiments, the instrumentation amplifier 214 is a chopper-stabilized instrumentation amplifier. Chopper-stabilized instrumentation amplifiers are widely used in precision measurement applications, high-precision sensor interfaces, and data acquisition systems, particularly in medical device applications and biosignal acquisition systems. These specialized amplifiers excel at amplifying low-frequency or DC signals with low noise and offset. The principle behind the operation of the chopper-stabilized instrumentation amplifier involves periodically switching or “chopping” the input signal at a high frequency, which modulates the input signal to a higher frequency where amplification can occur with reduced noise.

Feedback capacitors (C_{FB}) 208, in conjunction with input capacitors (C_{IN}) 202, set the mid-band gain of the instrumentation amplifier 214. The ratio of the input capacitors (C_{IN}) 202 to the feedback capacitors (C_{FB}) 208 determines the voltage gain, allowing control through well-matched on-chip capacitors.

High-pass capacitors (C_{HP}) 206, in tandem with the feedback integrator circuit 216, establish the high-pass characteristic of the AFE system 200, contributing to the overall frequency response of the instrumentation amplifier 214. The high-pass filtering is advantageous for rejecting electrode DC offsets and low-frequency drift, which could otherwise saturate the instrumentation amplifier 214.

The feedback integrator circuit 216 sets the high-pass corner frequency of the AFE system 200. By integrating the output and feeding it back through high-pass capacitors (C_{HP}) 206, the feedback integrator circuit 216 creates a controllable high-pass response. Depending on the desired accuracy and power consumption trade-offs, the feedback integrator circuit 216 may be implemented using switched-capacitor techniques or a continuous-time approach with pseudo-resistors.

The second switches (SW₂) 210 and the third switches (SW₃) 212 implement the chopping function, periodically reversing the polarity of the signal path. In embodiments, the chopping action occurs at a frequency above the signal band of interest, typically in the kHz range. In embodiments, the switching mechanism is controlled through a controller internal to the AFE system 200 or a processor external to the AFE system 200.

In embodiments, the output (OUT_{LNA}) of instrumentation amplifier 214 provides the amplified and filtered bio-signal to the LNA buffer circuit of the analog circuit 102 for further processing or digitization by the digital circuit 104. The overall transfer function of the AFE system 200 is a band-pass filter, with the low-frequency cutoff determined by the feedback integrator circuit 216 and the high-pass capacitors (C_{HP}) 206. The high-frequency cutoff is set by the inherent bandwidth of the instrumentation amplifier 214 or additional low-pass filtering (not shown).

FIG. 3 illustrates a schematic of an embodiment feedback integrator circuit 300, which may be implemented as the feedback integrator circuit 216 of the AFE system 200 of FIG. 2.

The feedback integrator circuit 300 includes a first circuit 302, an integration capacitor (C_{INT}) 304, and an amplifier 306, which may (or may not) be arranged as shown. Additional components or modifications may be included in the feedback integrator circuit 300 to enhance its performance and stability or to add specific functionalities, though these are not explicitly shown in the simplified schematic of FIG. 3.

Depending on the application, the first circuit 302 may be implemented using switched capacitors or pseudo-resistors. Switched capacitors can provide precise, tunable resistance-like behavior, while pseudo-resistors offer high resistance values in a compact form factor. The choice between these implementations can affect the circuit's frequency response and integration characteristics.

The integration capacitor (C_{INT}) 304 is coupled between the output and the inverting input of the amplifier 306. Integration capacitor (C_{INT}) 304 is employed for the integration function of the feedback integrator circuit 300, as it stores charge over time, performing the mathematical integration of the input signal.

In embodiments, amplifier 306 is depicted as an operational amplifier (op-amp) with a non-inverting input coupled to ground (or a reference voltage). The inverting input of the amplifier 306 is coupled to the output of the first circuit 302, while the output of the amplifier 306 forms the output of the feedback integrator circuit 300.

In operation, the first circuit 302 provides a controlled input to the integrator structure formed by the amplifier 306 and the integration capacitor (C_{INT}) 304. The amplifier 306, configured in the negative feedback arrangement with the integration capacitor (C_{INT}) 304, integrates the input signal over time.

The configuration allows the feedback integrator circuit 300 to perform continuous-time integration of the input signal, which is advantageous for analog signal processing applications, including the AFE system 200 in which it may be implemented.

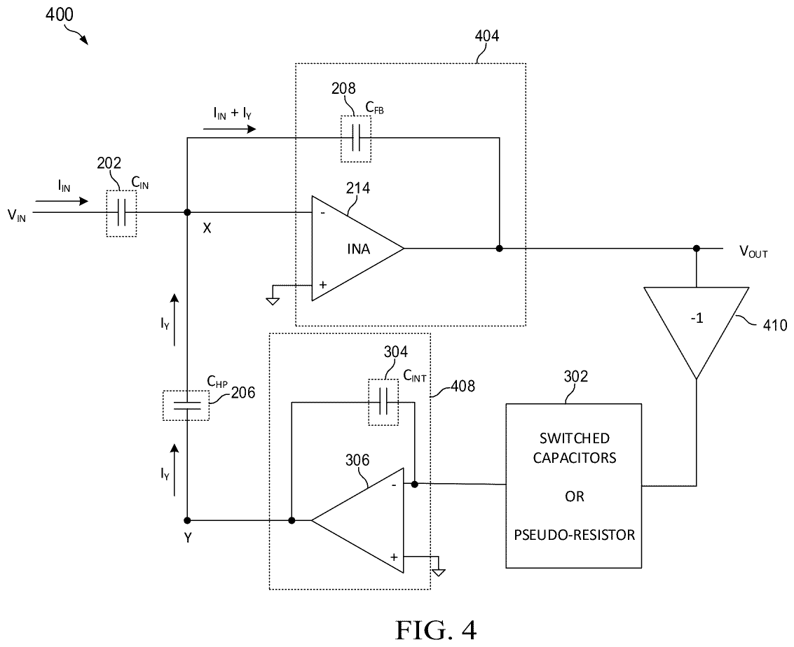

FIG. 4 illustrates a simplified schematic of an embodiment circuit 400. Circuit 400 is a streamlined representation of the AFE system 200 shown in FIG. 2. The simplified diagram retains the key functional components necessary to describe the operation and transfer functions of the analog front-end.

Circuit 400 includes the input capacitor (C_{IN}) 202, a forward amplifier 404, the high-pass capacitor (C_{HP}) 206, a feedback amplifier 408, the first circuit 302, and an inverting amplifier 410, which may or may not be arranged as shown. Forward amplifier 404 includes the feedback capacitor (C_{FB}) 208 and an instrumentation amplifier 214. Feedback amplifier 408 includes the integration capacitor (C_{INT}) 304 and the amplifier 306. The inverting amplifier 410 has a gain of −1 and is positioned at the output of the forward amplifier 404. The inverting amplifier 410 ensures that the feedback signal is in the correct phase for proper operation of the feedback loop. Components with similar element numbers are not described again for the sake of brevity.

First circuit 302, implemented as either switched capacitors or a pseudo-resistor, is retained from FIG. 3 to complete the feedback loop. The simplified version omits some of the more detailed circuitry and additional components in FIG. 2, such as the voltage references and specific filtering stages, while preserving the core signal path and feedback mechanism. This representation allows a more focused analysis of the AFE's primary operations and transfer characteristics.

A bandpass filtering architecture is commonly employed in bio-potential and bioimpedance measurement applications, such as ECG (Electrocardiogram and BIA (Bioelectrical Impedance Analysis).

The bandpass requirement is due to the nature of the measured signals and the requisite for optimizing signal quality while minimizing interference. For ECG signals, a bandpass filter with a range of 100 mHz to 300 Hz is required. This range typically captures the full spectrum of cardiac electrical activity while rejecting unwanted noise and interference. The lower cutoff of 100 mHz allows for the detection of slow variations in the ECG baseline, which can be advantageous for certain diagnostic purposes, while the upper limit of 300 Hz ensures that high-frequency components of the QRS complex are preserved.

For bioimpedance analysis (BIA) signals, the requirements are different due to the nature of the measurement technique. The system needs to operate at higher frequencies, typically above 30 kHz, to comply with safety standards and optimize measurement accuracy. The bandpass characteristics for BIA need to accommodate the injection of a current at these higher frequencies while also allowing for the detection of both the real and imaginary components of the measured voltage. This enables the system to evaluate electrode-skin contact, measure body impedance for defibrillation purposes, and detect AC components related to respiration and movement. The ability to configure the bandpass characteristics, especially the high-pass and low-pass cutoff frequencies, allows the system to be adaptable to different measurement scenarios and electrode types, enhancing its versatility and compliance with various commercial electrodes.

The architecture can be adapted for both ECG and BIA channels by adjusting the programmable gain and cut-off frequencies to suit the specific requirements of each measurement type. The bandpass characteristic can be achieved by combining a high-pass filter (HPF) and a low-pass filter (LPF).

In circuit 400, the low-pass frequency is primarily determined by the compensation of the forward amplifier 404, which also ensures the stability of circuit 400 and forms the upper limit of the passband. The high-pass capacitor (C_{HP}) 206, the integration capacitor (C_{INT}) 304, and the first circuit 302 sets the high-pass frequency.

For example, with a mid-band gain of 35/50 dB, a high-pass corner frequency of approximately 0.1 Hz, and a low-pass corner frequency of about 300 Hz, a low-pass frequency of approximately 300 Hz is primarily determined by the compensation of the forward amplifier 404. The feedback path sets the high-pass frequency of approximately 0.1 Hz.

The challenge in circuit 400 lies in achieving the very low high-pass corner frequency, such as 0.1 Hz. The low frequency is advantageous for accurately capturing low-frequency components of bio-potential signals, but it presents design difficulties, particularly regarding the required component values.

In embodiments where the first circuit 302 is implemented using a switched capacitor solution, the high-pass frequency (F_{HP}) is given by the equation:

$${F}_{INT} = {F}_{HP} = \frac{1}{2 π} \times \frac{{C}_{HP}}{{C}_{FB}} \times {F}_{SC} \times {C}_{SC} \times \frac{1}{{C}_{INT}} ,$$

where F_{SC} and C_{SC} are, respectively, frequency and capacitive values of the switched capacitor circuit.

In embodiments where the first circuit 302 is implemented using a pseudo-resistor solution, the high-pass frequency (F_{HP}) is given by the equation:

$${F}_{INT} = {F}_{HP} = \frac{1}{2 π} \times \frac{1}{R} \times \frac{{C}_{HP}}{{C}_{FB}} \times \frac{1}{{C}_{INT}} ,$$

where R is the equivalent resistor value of the pseudo-resistor circuit.

The AC gain (Gain_{AC}) follows the equation:

$$G a i {n}_{A C} = \frac{{OUT}_{A C}}{IN} = \frac{{C}_{IN}}{{C}_{FB}} = \frac{128 \times {C}_{0}}{g \times {C}_{0}} ,$$

where C_{IN} is the input capacitance, C_{FB} is the feedback capacitance, g is the gain factor, and C₀ is the base capacitance. The DC gain (Gain_{DC}) follows the equation:

$$G a i {n}_{D C} = \frac{{OUT}_{D C}}{IN} = \frac{{C}_{IN}}{{C}_{HP}} = \frac{128 \times {C}_{0}}{64 \times {C}_{0}} ,$$

where C_{HP} is the high-pass capacitance. The high-pass frequency (f_{HP}) follows the equation

$${f}_{HP} = \frac{{f}_{C}}{2 π} \times \frac{{C}_{HP}}{{C}_{FB}} \times \frac{{C}_{SC}}{{C}_{INT}} ,$$

where f_{C} is the chopping frequency, and C_{INT} is the integration capacitance.

To solve the value of the integration capacitance (C_{INT}), the equations above can be rearranged as:

$${C}_{INT} = \frac{{f}_{C}}{2 π} \times \frac{{C}_{HP}}{{C}_{FB}} \times \frac{{C}_{SC}}{{f}_{HP}} .$$

To illustrate the magnitude of the challenge, consider a numerical example for a bio-potential measurement. Assuming the base capacitance Cₒ is equal to 125 femtofarads (fF), the gain factor (g) is equal to 1, 2, 4, or 8, the chopping frequency is equal to 2 kHz, the input capacitance (C_{IN}) is equal to 128×Cₒ or 16 picofarads (pF), the high-pass capacitance (C_{HP}) is equal to 64×Cₒ or 8 picofarads (pF).

In this example, the AC gain (Gain_{AC}) is equal to 128 (i.e., approximately 42 dB) for a gain factor of 1 and 16 (i.e., approximately 24 dB) for a gain factor of 8, and the DC gain (Gain_{DC}) is equal to 2. To achieve a high-pass frequency (f_{HP}) of 0.1 Hz, the value of the feedback capacitance (C_{FB}) for a gain factor of 1 and a switched capacitance (C_{SC}) of 100 fF is equal to 125 fF, resulting in an integration capacitance (C_{INT}) value of 20.3 nF. A 20.3 nF capacitor is a large capacitance for on-chip integration.

Accordingly, for the switched capacitor and pseudo-resistor solutions, achieving the desired low high-pass frequency (F_{HP}) (e.g., approximately 0.1 Hz) necessitates a very large value for the integration capacitor (C_{INT}). Even at lower gain settings, the value of the integration capacitance (C_{INT}) remains in the nanofarads range.

The requirement presents significant challenges in integrated circuit design, such as chip area, manufacturing costs, parasitic effects, power consumption, and integration challenges. For example, capacitors in the nanofarads range consume a substantial amount of die area. In modern integrated circuits, where miniaturization is crucial, dedicating such a large area to a single component is often impractical and costly. Further, larger chip areas directly translate to higher manufacturing costs. The need for such large capacitors can significantly impact the overall cost-effectiveness of the design. Moreover, as capacitor size increases, so do associated parasitic effects, which can degrade performance and complicate the circuit design. Furthermore, larger capacitors typically lead to increased power consumption, which is particularly problematic in battery-operated or implantable medical devices where power efficiency is crucial.

A conventional approach to address the challenge of large integration capacitance in bio-potential measurement systems is using external capacitors. However, this approach comes with several significant drawbacks. First, it compromises the goal of a fully integrated solution, introducing additional complexity in assembly and potentially reducing overall reliability. Second, it results in a loss of flexibility in the design; adjusting the integrator's capacitance, which might be advantageous for different measurement scenarios or to accommodate varying electrode characteristics, would require physical replacement of external components rather than simple reconfiguration via on-chip digital controls. This complicates system updates and burdens such modifications on the end-user or customer. Further, the need for external capacitors increases the overall bill of materials and system cost, potentially making the solution less competitive in price-sensitive markets.

Embodiments of this disclosure propose a fully integrated solution to address the shortcomings in the conventional solutions with integration capacitance (C_{INT}) values in the picofarad (pF) range.

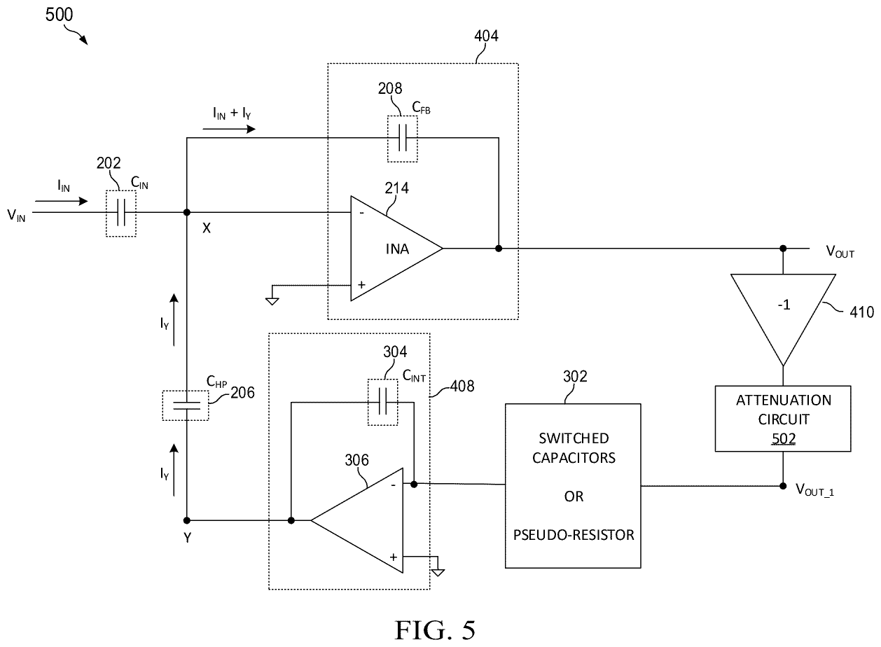

FIG. 5 illustrates a simplified schematic of an embodiment circuit 500. Circuit 500 is a streamlined representation of the AFE system 200 shown in FIG. 2 with the addition of an attenuation circuit 502. In embodiments, the attenuation circuit 502 is a passive attenuator, an active attenuator, or a passive attenuator with an active control mechanism.

In embodiments, the attenuation circuit 502 can be constructed using, for example, a passive attenuator, such as a resistor divider network with two resistors coupled in series between the output (V_{OUT}) of the forward amplifier 404 and ground. The junction point between the resistors provides the attenuated feedback signal V_{OUT_1}. The ratio of the resistors determines the attenuation factor A. For instance, if the first resistor (R₁) of the attenuation circuit 502 is the resistor coupled to V_{OUT} and the second resistor (R₂) is coupled to ground, the attenuation factor A would be equal to

$$\frac{{R}_{1} + {R}_{2}}{{R}_{2}} .$$

In embodiments, the attenuation circuit 502 is constructed using, for example, an active attenuator, such as a resistive-capacitive network, a voltage divider followed by a unity-gain buffer amplifier, or a switched-capacitor circuit.

In an implementation, the attenuation circuit 502 may include an operational amplifier in an inverting configuration, where the ratio of the feedback resistor to the input resistor determines the attenuation factor. For instance, if the input resistor is R₁ and the feedback resistor is R₂ (where R₂<R₁), the attenuation factor would be R₁/R₂.

In embodiments, the attenuation circuit 502 can be implemented as a voltage divider followed by a unity-gain buffer amplifier. The configuration can provide the desired attenuation while maintaining high input and low output impedances. The voltage divider sets the attenuation factor, while the buffer amplifier prevents loading effects on subsequent stages.

In embodiments, the attenuation circuit 502 includes a switched-capacitor circuit. This approach can offer advantages in terms of precision and programmability. The attenuation factor can be accurately set and potentially adjusted dynamically during operation by controlling the switching frequency and capacitor ratios.

In embodiments, the attenuation circuit 502 is implemented using a digital potentiometer or a digitally controlled analog attenuator. The configuration allows for software-controlled adjustment of the attenuation factor, providing flexibility in system calibration and adaptation to different measurement conditions.

In embodiments, the implementation of attenuation circuit 502 can be made based on practical factors associated with the bio-potential measurement. For example, factors such as noise, input impedance, and frequency response can be used to select the implementation type for the attenuator circuit 502.

The operation of circuit 500 can be understood through a series of equations that describe the relationships between various voltages and currents in the system. Starting with the input stage, we can express the relationship between the input voltage (VIN) and input current (I_{IN}) as:

$${V}_{IN} = {I}_{IN} \times \frac{1}{s \times {C}_{IN}} ,$$

where s is the complex frequency variable and C_{IN} is the input capacitor (C_{IN}) 202. The feedback path, which includes the integration capacitance (C_{INT}) 304 and the attenuation circuit 502, can be represented by the equation:

$${V}_{Y} = - \frac{{V}_{O U T}}{s \times R \times {C}_{INT}} ,$$

where V_{Y} is the voltage at node Y, R is the equivalent resistance of the feedback path, and C_{INT} is the integration capacitance (C_{INT}) 304.

The relationship between the current I_{Y} flowing through the high-pass capacitor (C_{HP}) 206 and the voltage V_{Y} at node Y is given by the equation:

$${V}_{Y} = {I}_{Y} \times \frac{1}{s \times {C}_{H P}} .$$

The forward amplifier's operation, including the effect of the high-pass capacitor (C_{HP}) 206, can be expressed as:

$${V}_{O U T} = - ( {I}_{Y} + {I}_{IN} ) \times \frac{1}{sC} = - \frac{{I}_{Y}}{sC} - \frac{{I}_{IN}}{sC} ,$$

where C is the feedback capacitance of the forward amplifier 404.

Through a series of substitutions and algebraic manipulations, we can derive the overall transfer function of the circuit:

$${V}_{O U T} = \frac{s \frac{R \times {C}_{INT}}{{C}_{H P}} \times \frac{{C}_{IN}}{C}}{1 - \frac{R \times {C}_{INT}}{{C}_{H P}} s C} .$$

The overall transfer function equation illustrates that circuit 500 behaves as a high-pass filter, with the cutoff frequency and gain determined by the ratios of the various capacitances and the feedback resistance. Notably, the low-frequency behavior is not visible in the transfer function, demonstrating that the circuit effectively blocks DC and very low-frequency components of the input signal.

A benefit of the modification is the ability to substantially reduce the size of the integration capacitor (C_{INT}) 304. By attenuating the feedback signal, the effective gain of the feedback loop is reduced by a factor of A.

In embodiments where the first circuit 302 is implemented using a switched capacitor solution, the high-pass frequency (F_{HP}) is given by the updated equation:

$${F}_{HP} = \frac{{f}_{C}}{2 π} \times \frac{{C}_{HP}}{{C}_{FB}} \times \frac{{C}_{SC}}{{C}_{INT}} \times \frac{1}{A} ,$$

where A is the attenuation factor.

In embodiments where the first circuit 302 is implemented using a pseudo-resistor solution, the high-pass frequency (F_{HP}) is given by the updated equation:

$${F}_{HP} = \frac{1}{2 π} \times \frac{1}{R} \times \frac{{C}_{HP}}{{C}_{FB}} \times \frac{1}{{C}_{INT}} .$$

Accordingly, to maintain the same overall transfer function and frequency response, the capacitance of the integration capacitor (C_{INT}) 304 can be decreased by the same factor. The practical impact of this modification is substantial.

For bioimpedance analysis (BIA) mode, an attenuation factor of 5 can be employed, while for electrocardiogram (ECG) mode, a factor of 20 is suitable. The attenuation circuit 502 can be implemented using, for example, a network of switchable resistors, a digitally controlled potentiometer, or an active attenuator, such as a resistive-capacitive network, to achieve the desired flexibility in attenuation factors based on the operating mode. This allows for dynamic adjustment of the attenuation factor based on the operating mode (ECG or BIA) or the phase of operation (startup or normal running). The switching mechanism can be controlled by digital logic, enabling easy integration with the overall system control.

In some cases, the attenuation factor can reach values of several hundred. These large attenuation factors allow for a dramatic reduction in the required capacitance of the integration capacitor (C_{INT}) 304, potentially decreasing it by two orders of magnitude from the nanofarads range to approximately 10 picofarads. The reduction in capacitor size translates directly to significant chip area savings, especially when implemented across multiple channels in a device.

In embodiments, the attenuation factor is determined in accordance with the noise in the feedback loop. As the attenuation factor is increased, resulting in a smaller capacitance for the integration capacitor (C_{INT}) 304, the noise in the feedback loop increases. Accordingly, a balance is struck between the selection of the attenuation factor and maintaining the noise level in the feedback loop within a reasonable value.

Further, the approach introduces flexibility into the design. The same basic circuit structure can be used for both ECG and BIA channels, with the only difference being the chosen attenuation ratio. The versatility extends to the channel's operational phases as well. Using simple control bits, the attenuation factor can be dynamically adjusted during different stages of operation, such as startup and normal functioning, allowing for optimized performance in each phase.

The implications of the design modification extend beyond just area savings. Smaller capacitors can improve overall circuit performance. Additionally, the ability to fine-tune the circuit's characteristics through the attenuation factor provides designers with a powerful tool for optimizing the analog front-end for specific applications or operating conditions.

For the integration capacitor (C_{INT}) 304, the reduced capacitance requirement allows for the use of high-quality, on-chip capacitors. The integration capacitor (C_{INT}) 304 can be implemented using metal-insulator-metal (MIM) capacitors or other specialized capacitor structures available in modern CMOS processes.

The first circuit 302 interfacing with the attenuated feedback signal can be modified to accommodate the reduced signal levels. This may involve adjusting the sizing of the switches and capacitors in the switched-capacitor implementation or tuning the characteristics of the pseudo-resistor to maintain the desired frequency response with the attenuated signal.

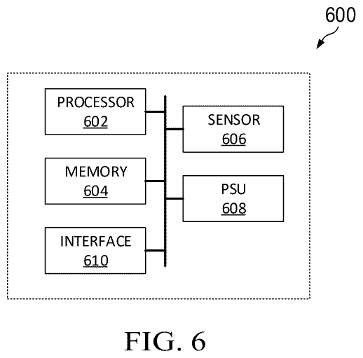

FIG. 6 illustrates a block diagram of an embodiment medical device 600 for biosignal measurements. In embodiments, medical device 600 may be used to collect or display measurements such as an electrocardiogram (ECG), photoplethysmogram (PPG), bioelectrical impedance analysis (BIA) signal, or galvanic skin response/electrodermal activity (GSR/EDA) signal.

Medical device 600 includes a processor 602, a memory 604, a sensor 606, a power supply unit (PSU) 608, and an interface 610, which may (or may not) be arranged as shown. Although one of each (i.e., the processor 602, the memory 604, the sensor 606, the power supply unit 608, and the interface 610) is shown in FIG. 6, the number of components is not limiting, and greater numbers are similarly contemplated in other embodiments.

Medical device 600 may include additional components not depicted, such as long-term storage (e.g., non-volatile memory, etc.), power management circuitry, security and encryption modules (e.g., trusted platform modules (TPM), etc.), or the like.

Medical device 600 may be an electronic device, such as a smartwatch, fitness tracker, medical device (e.g., pulse oximeters), wristband, sports band, smart ring, earbuds, or any device capable of hosting the sensor 606.

In embodiments, each component can communicate with any other component internally within or external to the medical device 600. For example, each component can communicate using the I2C (Inter-Integrated Circuit), alternatively known as I2C or IIC, communication protocol, the 13C (Improved Inter Integrated Circuit) communication protocol, the serial peripheral interface (SPI) specification, or the like.

Processor 602 may be any component or collection of components adapted to perform computations or other processing-related tasks. In embodiments, processor 602 is a host processor, an application processor, a baseband processor, or a microcontroller. In embodiments, processor 602 is configured to control the operation of the medical device 600.

Memory 604 may be any component or collection of components adapted to store programming, instructions, or calibration settings for execution or retrieval by processor 602. In an embodiment, memory 604 includes a non-transitory computer-readable medium.

Sensor 606 may be any component or collection of components adapted for biosignal measurements. Sensor 606 may include the medical sensor 100 of FIG. 1. In addition, sensor 606 may include one or more sensing components for biosignal measurements. For example, sensor 606 may include a PPG sensor, a BIA sensor, a GSR/EDA sensor, or a combination thereof, coupled to the medical sensor 100 of FIG. 1. In embodiments, sensor 606 may include one or more accelerometers, gyroscopes, and inertial measurement units (IMUs).

For example, sensor 606 may include an integrated emitter and detector for PPG measurements. In embodiments, sensor 606 is an integrated solution comprising the medical sensor 100 and various sensing components. Sensor 606 may include additional components not shown, such as memory, a dedicated microcontroller, and a driver.

It should be noted that medical device 600 may include additional sensing components (e.g., detector, emitter, etc.) for collecting the bio-signals and PPG signals that are external to an integrated solution. In such embodiments, sensor 606 is coupled to the external sensing components to receive the data as they are collected.

In embodiments, sensor 606 is configured to receive a differential photodiode signal from a detector, generate compensation currents for the DC and ambient light components of the electrical signal, amplify the electrical signal, isolate the PPG signal from unrelated signals or noise to improve the signal-to-noise ratio, and process the PPG signals.

In embodiments, sensor 606 is configured to receive a differential biopotential signal from a detector, amplify the electrical signal, isolate the biopotential signal from unrelated signals or noise to improve the signal-to-noise ratio, and process the biosignal.

Power supply unit 608 may be any component or collection of components that provide power to one or more components within the medical device 600. Power supply unit 608 may include various power management circuitry, charge storage components (i.e., battery), or the like.

Interface 610 may be any component or collection of components that allow processor 602 to communicate with other devices/components or a user.

In embodiments, data collected and processed by sensor 606 is stored in memory 604. In embodiments, processor 602 further processes the processed data by sensor 606 to be uploaded to the cloud, displayed on interface 610, or the like.

In embodiments, processor 602 receives data from sensor 606, interprets it, and converts it into usable biometric information, such as heart rate, heart rate variability, blood oxygen saturation (SpO2), and blood pressure trends. In embodiments, processor 602 is configured to alert a user of an anomaly related to the biosignal measurement through interface 610. Processor 602 may apply signal processing algorithms to refine the data, compensating for factors like ambient light noise or object reflectivity variations to provide more reliable information.

A first aspect relates to a biopotential measurement system, comprising a sensing electrode configured to detect a biopotential signal; an analog front-end circuit coupled to the sensing electrode, the analog front-end circuit comprising a forward amplifier configured to amplify the biopotential signal; a feedback amplifier configured to integrate an output of the forward amplifier and set a high-pass corner frequency of the analog front-end circuit, the feedback amplifier comprising an integration capacitor and an amplifier; an attenuation circuit coupled between an output of the forward amplifier and an input of the feedback amplifier, wherein the attenuation circuit provides an attenuation factor that determines a capacitance value of the integration capacitor for achieving the high-pass corner frequency; and a digital circuit coupled to the analog front-end circuit, the digital circuit comprising a processor configured to process the amplified biopotential signal.

In a first implementation form of the biopotential measurement system, according to the first aspect as such, the attenuation circuit is configurable to provide different attenuation factors for different types of biopotential signals.

In a second implementation form of the biopotential measurement system, according to the first aspect as such or any preceding implementation form of the first aspect, the attenuation circuit comprises a network of switchable resistors, and wherein the attenuation factor is configurable by setting the network of switchable resistors.

In a third implementation form of the biopotential measurement system, according to the first aspect as such or any preceding implementation form of the first aspect, wherein a ratio of resistances in the attenuation circuit determines the attenuation factor of the attenuation circuit.

In a fourth implementation form of the biopotential measurement system, according to the first aspect as such or any preceding implementation form of the first aspect, the biopotential signal is an electrocardiogram (ECG) signal, a bioelectrical impedance analysis (BIA) signal.

In a fifth implementation form of the biopotential measurement system, according to the first aspect as such or any preceding implementation form of the first aspect, the analog front-end circuit further comprises a circuit arranged between the attenuation circuit and a common node between an inverting input of the amplifier of the feedback amplifier and the integration capacitor, the circuit comprising switched capacitors or a pseudo-resistor, wherein the circuit is configured to provide a controlled input to the feedback amplifier for continuous-time integration of the output of the forward amplifier.

In a sixth implementation form of the biopotential measurement system, according to the first aspect as such or any preceding implementation form of the first aspect, the analog front-end circuit further comprises an input capacitor, wherein the forward amplifier comprises a feedback capacitor, and wherein a ratio of the feedback capacitor to the input capacitor sets a mid-band gain of the analog front-end circuit.

A second aspect relates to an analog front-end circuit for biopotential measurement, the analog front-end circuit comprising a forward amplifier configured to amplify a biopotential signal; a feedback amplifier configured to integrate an output of the forward amplifier and set a high-pass corner frequency of the analog front-end circuit, the feedback amplifier comprising an integration capacitor and an amplifier; an attenuation circuit coupled between an output of the forward amplifier and an input of the feedback amplifier, wherein the attenuation circuit provides an attenuation factor that determines a capacitance value of the integration capacitor for achieving the high-pass corner frequency; and a circuit arranged between the attenuation circuit and a common node between an inverting input of the amplifier of the feedback amplifier and the integration capacitor, the circuit comprising switched capacitors or a pseudo-resistor.

In a first implementation form of the analog front-end circuit, according to the second aspect as such, the analog front-end circuit further comprises a high-pass capacitor coupled between an input of the forward amplifier and an output of the feedback amplifier, wherein the high-pass capacitor and the feedback amplifier establish a high-pass characteristic of the analog front-end circuit.

In a second implementation form of the analog front-end circuit, according to the second aspect as such or any preceding implementation form of the second aspect, the forward amplifier is a chopper-stabilized instrumentation amplifier.

In a third implementation form of the analog front-end circuit, according to the second aspect as such or any preceding implementation form of the second aspect, the forward amplifier comprises an inherent bandwidth limitation that determines an upper cutoff frequency of the analog front-end circuit, and wherein the feedback amplifier determines a lower cutoff frequency, thereby forming a bandpass response for the analog front-end circuit.

In a fourth implementation form of the analog front-end circuit, according to the second aspect as such or any preceding implementation form of the second aspect, the integration capacitor has a capacitance value in the picofarad range to provide high-pass filtering with a cutoff frequency of 100 mHz.

In a fifth implementation form of the analog front-end circuit, according to the second aspect as such or any preceding implementation form of the second aspect, the analog front-end circuit further comprising a multiplexer coupled to an input of the forward amplifier and configured to selectively route different biopotential signals to the forward amplifier.

In a sixth implementation form of the analog front-end circuit, according to the second aspect as such or any preceding implementation form of the second aspect, the attenuation circuit is dynamically adjustable to modify the attenuation factor during operation of the analog front-end circuit.

A third aspect relates to a biopotential measurement system, comprising a first analog front-end circuit configured to process a first biopotential signal, the first analog front-end circuit comprising a first forward amplifier, a first feedback amplifier comprising a first integration capacitor and a first amplifier, and a first attenuation circuit coupled between an output of the first forward amplifier and an input of the first feedback amplifier, wherein the first attenuation circuit provides a first attenuation factor, a second analog front-end circuit configured to process a second biopotential signal, the second analog front-end circuit comprising a second forward amplifier, a second feedback amplifier comprising a second integration capacitor and a second amplifier, and a second attenuation circuit coupled between an output of the second forward amplifier and an input of the second feedback amplifier, wherein the second attenuation circuit provides a second attenuation factor; and a digital circuit comprising a processor coupled to outputs of the first and second analog front-end circuits, the processor configured to dynamically adjust the first and second attenuation factors based on characteristics of the first and second biopotential signals.

In a first implementation form of the biopotential measurement system, according to the third aspect as such, the first biopotential signal is an electrocardiogram (ECG) signal and the second biopotential signal is a bioelectrical impedance analysis (BIA) signal, and wherein the first and second attenuation factors are different from each other.

In a second implementation form of the biopotential measurement system, according to the third aspect as such or any preceding implementation form of the third aspect, the biopotential measurement system further comprising a multiplexer arranged between the digital circuit and the first and second analog front-end circuits, the multiplexer configured to selectively route different biopotential signals from the first and second analog front-end circuits to the digital circuit based on instructions from the processor.

In a third implementation form of the biopotential measurement system, according to the third aspect as such or any preceding implementation form of the third aspect, the processor is further configured to adjust the first and second attenuation factors in accordance with a biopotential signal type of the first and second biopotential signal.

In a fourth implementation form of the biopotential measurement system, according to the third aspect as such or any preceding implementation form of the third aspect, the processor is configured to simultaneously process the first and second biopotential signals to provide multi-parameter physiological monitoring.

In a fifth implementation form of the biopotential measurement system, according to the third aspect as such or any preceding implementation form of the third aspect, the processor is configured to analyze the first and second biopotential signals to detect anomalies and to generate an alert based thereon.

Although the description has been described in detail, it should be understood that various changes, substitutions, and alterations may be made without departing from the spirit and scope of this disclosure as defined by the appended claims. The same elements are designated with the same reference numbers in the various figures. Moreover, the scope of the disclosure is not intended to be limited to the particular embodiments described herein, as one of ordinary skill in the art will readily appreciate from this disclosure that processes, machines, manufacture, compositions of matter, means, methods, or steps, presently existing or later to be developed, may perform substantially the same function or achieve substantially the same result as the corresponding embodiments described herein. Accordingly, the appended claims are intended to include within their scope such processes, machines, manufacture, compositions of matter, means, methods, or steps.

The specification and drawings are, accordingly, to be regarded simply as an illustration of the disclosure as defined by the appended claims, and are contemplated to cover any and all modifications, variations, combinations, or equivalents that fall within the scope of the present disclosure.

What is claimed is:

1. A biopotential measurement system, comprising:
a sensing electrode configured to detect a biopotential signal;
an analog front-end circuit coupled to the sensing electrode, the analog front-end circuit comprising:
a forward amplifier configured to amplify the biopotential signal;
a feedback amplifier configured to integrate an output of the forward amplifier and set a high-pass corner frequency of the analog front-end circuit, the feedback amplifier comprising an integration capacitor and an amplifier;
an attenuation circuit coupled between an output of the forward amplifier and an input of the feedback amplifier, wherein the attenuation circuit provides an attenuation factor that determines a capacitance value of the integration capacitor for achieving the high-pass corner frequency; and
a digital circuit coupled to the analog front-end circuit, the digital circuit comprising a processor configured to process the amplified biopotential signal.

2. The biopotential measurement system of claim 1, wherein the attenuation circuit is configurable to provide different attenuation factors for different types of biopotential signals.

3. The biopotential measurement system of claim 1, wherein the attenuation circuit comprises:
a passive attenuator having a network of switchable resistors, wherein the attenuation factor is configurable by setting the network of switchable resistors, or
an active attenuator having an operational amplifier in inverting configuration, a voltage divider with a unity-gain buffer amplifier, a switched capacitor circuit, or a digitally controlled analog attenuator or digital potentiometer.

4. The biopotential measurement system of claim 1, wherein a ratio of resistances in the attenuation circuit determines the attenuation factor of the attenuation circuit.

5. The biopotential measurement system of claim 1, wherein the biopotential signal is an electrocardiogram (ECG) signal, a bioelectrical impedance analysis (BIA) signal.

6. The biopotential measurement system of claim 1, wherein the analog front-end circuit further comprises a circuit arranged between the attenuation circuit and a common node between an inverting input of the amplifier of the feedback amplifier and the integration capacitor, the circuit comprising switched capacitors or a pseudo-resistor, wherein the circuit is configured to provide a controlled input to the feedback amplifier for continuous-time integration of the output of the forward amplifier.

7. The biopotential measurement system of claim 1, wherein the analog front-end circuit further comprises an input capacitor, wherein the forward amplifier comprises a feedback capacitor, and wherein a ratio of the feedback capacitor to the input capacitor sets a mid-band gain of the analog front-end circuit.

8. An analog front-end circuit for biopotential measurement, the analog front-end circuit comprising:
a forward amplifier configured to amplify a biopotential signal;
a feedback amplifier configured to integrate an output of the forward amplifier and set a high-pass corner frequency of the analog front-end circuit, the feedback amplifier comprising an integration capacitor and an amplifier;
an attenuation circuit coupled between an output of the forward amplifier and an input of the feedback amplifier, wherein the attenuation circuit provides an attenuation factor that determines a capacitance value of the integration capacitor for achieving the high-pass corner frequency; and
a circuit arranged between the attenuation circuit and a common node between an inverting input of the amplifier of the feedback amplifier and the integration capacitor, the circuit comprising switched capacitors or a pseudo-resistor.

9. The analog front-end circuit of claim 8, further comprising a high-pass capacitor coupled between an input of the forward amplifier and an output of the feedback amplifier, wherein the high-pass capacitor and the feedback amplifier establish a high-pass characteristic of the analog front-end circuit.

10. The analog front-end circuit of claim 8, wherein the forward amplifier is a chopper-stabilized instrumentation amplifier.

11. The analog front-end circuit of claim 8, wherein the forward amplifier comprises an inherent bandwidth limitation that determines an upper cutoff frequency of the analog front-end circuit, and wherein the feedback amplifier determines a lower cutoff frequency, thereby forming a bandpass response for the analog front-end circuit.

12. The analog front-end circuit of claim 8, wherein the integration capacitor has a capacitance value in the picofarad range to provide high-pass filtering with a cutoff frequency of 100 mHz.

13. The analog front-end circuit of claim 8, further comprising a multiplexer coupled to an input of the forward amplifier and configured to selectively route different biopotential signals to the forward amplifier.

14. The analog front-end circuit of claim 8, wherein the attenuation circuit is dynamically adjustable to modify the attenuation factor during operation of the analog front-end circuit.

15. A biopotential measurement system, comprising:
a first analog front-end circuit configured to process a first biopotential signal, the first analog front-end circuit comprising:
a first forward amplifier,
a first feedback amplifier comprising a first integration capacitor and a first amplifier, and
a first attenuation circuit coupled between an output of the first forward amplifier and an input of the first feedback amplifier, wherein the first attenuation circuit provides a first attenuation factor,
a second analog front-end circuit configured to process a second biopotential signal, the second analog front-end circuit comprising:
a second forward amplifier,
a second feedback amplifier comprising a second integration capacitor and a second amplifier, and
a second attenuation circuit coupled between an output of the second forward amplifier and an input of the second feedback amplifier, wherein the second attenuation circuit provides a second attenuation factor; and
a digital circuit comprising a processor coupled to outputs of the first and second analog front-end circuits, the processor configured to dynamically adjust the first and second attenuation factors based on characteristics of the first and second biopotential signals.

16. The biopotential measurement system of claim 15, wherein the first biopotential signal is an electrocardiogram (ECG) signal and the second biopotential signal is a bioelectrical impedance analysis (BIA) signal, and wherein the first and second attenuation factors are different from each other.

17. The biopotential measurement system of claim 15, further comprising a multiplexer arranged between the digital circuit and the first and second analog front-end circuits, the multiplexer configured to selectively route different biopotential signals from the first and second analog front-end circuits to the digital circuit based on instructions from the processor.

18. The biopotential measurement system of claim 15, wherein the processor is further configured to adjust the first and second attenuation factors in accordance with a biopotential signal type of the first and second biopotential signal.

19. The biopotential measurement system of claim 15, wherein the processor is configured to simultaneously process the first and second biopotential signals to provide multi-parameter physiological monitoring.

20. The biopotential measurement system of claim 15, wherein the processor is configured to analyze the first and second biopotential signals to detect anomalies and to generate an alert based thereon.
